(19) United States
(12) Patent Application Publication
(10) Pub. No.: US 2023/0015932 A1
(43) Pub. Date: Jan. 19, 2023

(54) METHOD OF GENERATION OF LYMPHO-MYELOID NICHES

(71) Applicant: Natesan Senthilkumar, Gandhinagar (IN)

(72) Inventor: Natesan Senthilkumar, Gandhinagar (IN)

(21) Appl. No.: 17/786,188

(22) PCT Filed: Jul. 5, 2020
(86) PCT No.: PCT/IB2020/056315

§ 371 Date: Jun. 16, 2022

(30) Foreign Application Priority Data
Dec. 17, 2019 (IN) 201921052318

(51) Int. Cl. C12N 5/0783; C12N 5/0786

(52) U.S. Cl. CPC C12N 5/0636; C12N 5/0645; C12N 2506/11; C12N 2501/2302; C12N 2509/00; C12N 2501/2307; C12N 2501/2315; C12N 2501/2304

(57) ABSTRACT

The present invention relates to novel method of generating “Lympho-Myeloid Niches (LMN)” from peripheral blood mononuclear cell (PBMC). The present invention relates to a method of generating macrophages, myeloid cells and T cell from Lympho-Myeloid Niches (LMN). The present invention also describes its application for developing novel cell based therapies, gene therapies, gene edited therapies for the treatment of various disease conditions using the Lympho-Myeloid Niches (LMN), and/or the cells generated from Lympho-Myeloid Niches (LMN) or their culture.

### FIELD OF THE INVENTION

The present invention describes a novel method of generating “Lympho-Myeloid Niches (LMN)” from peripheral blood mononuclear cell (PBMC) obtained from human blood. The invention describes the developmental biology of Lympho-Myeloid Niches (LMN) and its application. The present invention relates to a novel method of generating macrophages or myeloid cells and novel method of generating T cell from Lympho-Myeloid Niches (LMN). The present invention also describes its application for developing novel cell based therapies or gene therapies or gene edited therapies for the treatment of various disease conditions using the Lympho-Myeloid Niches (LMN), and/or the T cells or myeloid cells generated from Lympho-Myeloid Niches (LMN) or their culture, use of these cells for gene therapies or gene edited therapies including Chimeric Antigen Receptor (CAR) T cell therapies or other adoptive T cell immunotherapies, for understanding any disease pathophysiology, creating novel animal models, development of novel drugs, developing vaccines or diagnostics or treatment including but not limited to Human immunodeficiency virus, cancer, Diabetic foot ulcers, non-healing wounds, autoimmune diseases, infectious diseases, etc.

### BACKGROUND OF THE INVENTION

Developmental biology of human immune cells is highly complex and there is still a lot to understand. In particular, the T cell generation in human is still poorly understood area and all our current understanding of T cell and most other immune cell development are based on the studies done in mice and its relevance to human is questionable. Most T cell developmental studies were carried out using the thymus and extra-thymic T cell development is also poorly understood, even though there are strong evidences for extra-thymic T cell development in human following surgical removal of thymus. Understanding the T cell progenitor that emigrate bone marrow and travel in the blood circulation prior to reach thymus when thymus is present or elsewhere when thymus is absent. For studying the T cell development in human, we need a simple and reliable in vitro model which could help us to understand the HIV and other infectious diseases as well as for developing novel gene therapy approaches through genome editing methods for cancers and genetic disorders.

The T cells can be obtained from methods available in art and it can be derived or obtained from many sources known in the art. For example, T cells can be differentiated in vitro from a stem cell population using foetal thymic organ culture (FTOC) model system. This approach relies on the seeding of human hematopoietic stem cells (HSCs) and/or their progeny into host thymic lobes or thymic fragments, typically of mouse origin. An in vitro approach that makes use of the OP9 bone marrow stromal cell line expressing the Notch receptor ligand Delta-like-1 (OP9-DL1) have also been shown to support the generation of large numbers of human progenitor T cells from HSCs. These methods of T cell generation may not reflect the natural mechanisms of T cell generation of in the human body.

The donor can be a subject, e.g., a subject in need of an anti-cancer treatment or wound healing treatment. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more blood cells or fractions available in the art. T cells can also be obtained from a unit of blood collected from a subject using any number of techniques known to the skilled artisan, such as FICOLL™ separation and/or apheresis. T cells can also be obtained from an artificial thymic organoid (ATO) cell culture system, which replicates the human thymic environment to support efficient ex vivo differentiation of T-cells from primary and reprogrammed pluripotent stem cells. Additional methods of isolating T cells for a T cell therapy are disclosed in many other patents, which is herein incorporated by references in its entirety.

U.S. Pat. No. 5,504,003A disclosed a human macrophage inflammatory protein-3 (MIP-3) and a human macrophage inflammatory protein-4 (MIP-4) polypeptides and DNA (RNA) encoding such polypeptides. There is also provided a procedure for producing such polypeptides by recombinant techniques and for producing antibodies against such polypeptides. In the invention, there is also provided antagonist/inhibitors against such polypeptides which inhibit the functioning of such polypeptides.

WO2010030947A1 describes a system and method for producing T cells from stem cell populations. Specifically exemplified herein is a culture system and method that produces CD4 cells and/or T cell subtypes from a CD4 lineage using a sample of hematopoietic stem cells. Adult hematopoietic precursor/stem cells (HPC) are progenitors to all lineages of immune cells.

EP2853590B1 discloses method for producing human CD8 single-positive cells or CD4 single-positive cells having antigen specificity, wherein the method comprises a step for differentiating to CD4/CD8 double-negative cells iPS cells induced from human T cells, a step for stimulating the T cell receptor of the CD4/CD8 double-negative cells, and a step for differentiating to CD8 single-positive or CD4 single-positive cells the CD4/CD8 double-negative cells whose T-cell receptor has been stimulated.

WO1996022781A1 a discloses method for preparing a composition containing optionally activated macrophages, and/or cells derived from monocytes having a high antigen presentation potential, wherein the monocytes in the starting composition are cultured, this step being preceded and/or followed by removal of at least some of the components other than monocytes from the starting composition, by means of antibodies to such components, and/or followed by elutriation. Compositions and kits for carrying out the method are also disclosed.

US20180298337 relates to a method for inducing CD4-positive T cells, which method comprises a step of introducing a CD4 gene or a gene product thereof into T cells induced from pluripotent stem cells. It also discloses production of CD4-positive T cells from pluripotent stem cells. Another object of the present invention is activation of dendritic cells using CD4-positive T cells obtained by this method.

U.S. Pat. No. 5,866,115A describes a process in which peripheral blood cells are first isolated and the CD 34 antigen-expressing blood progenitor cells which they contain are then enriched. These enriched cells are expanded ex-vivo using a combination of haematopoietic growth factors and cytokines. Over a period of 10-20 days, they give rise, in particular to dendritic cells which, where appropriate, can be purified still further. These cells are functionally active with regard to the ability to present antigen.

WO2014039044A1 describes a methods of producing an isolated T memory stem cell population, the method comprising a) isolating nave T cells from a mammal, wherein the mammal is not a mouse; b) activating the nave T cells and expanding the numbers of nave T cells in the presence of one or more non-specific T cell stimuli, one or more cytokines, and a GSK-3beta inhibitor.

However, till date there is no any disclosure of any method or process available to generate Lympho-Myeloid Niche from the peripheral blood mononuclear cells (PBMC) from the cultured peripheral blood mononuclear cells or to generate T cells or macrophages from them in a simple and cost-effective way.

Inventor of present invention have invented novel peripheral blood mononuclear cell derived Niches having capacity to generate T cells and macrophages. The invention can be used for generating T cells and macrophages efficiently in an easy and simple way as described herein.

### SUMMARY OF THE INVENTION

The present invention relates to a novel method of generating Lympho-Myeloid Niches (LMN) from peripheral blood mononuclear cells and its application thereof.

The present invention also relates process of preparing the macrophages and myeloid cells as well as T Cells from LMN.

One aspect of the present invention is to provide Lympho-Myeloid Niches (LMN) generated in vitro from PBMC which comprises of aggregation of cells and forming colony structures harbouring mainly T cells and macrophages, wherein LMN is characterized by the following features;
a) LMN provides a microenvironment, where the lymphoid and myeloid cells interact to each other and with the progenitor cells to de novo generate T cells and macrophages,
b) LMN are spontaneously generated in the cultures of peripheral blood mononuclear cells,
c) LMN are adherent to the culture vessels and remain intact upon removal of nonadherent cells,
d) LMN can de novo generate T cells and macrophages, where the newly generated T cells and macrophages migrate from the centre of the lympho myeloid niche towards the periphery,
e) LMN can be induced to generate T cells and macrophages by providing cytokines or growth factors that promote the T cells and macrophage proliferation, differentiation, and expansion.

The main aspect of the present invention is to provide a method of generating Lympho-Myeloid Niches (LMN) from peripheral blood mononuclear cells.

Yet another aspect of the present invention is to provide a method of generating T cells and macrophages from Lympho-Myeloid Niches (LMN).

As per one aspect, the present invention is to provide a method of generating Lympho-Myeloid Niches (LMN) comprising the steps of
a) Collecting blood from donor in a collection tube or bag containing anti-coagulant,
b) Recovering or isolating the PBMC from blood through Ficoll gradient centrifugation or any other method,
c) Resuspending the final cell pellet in Culture medium containing 20% fetal bovine serum as growth medium,
d) Counting and seeding PBMC in cell culture flask or vessel,
e) Incubating the culture flask or vessel at 37° C. temperature in the presence of 5% CO2,
f) Observing the generation of adherent Lympho-Myeloid Niches (LMN) under the inverted microscope,
g) Removing nonadherent cells from culture flask or vessel by gently shaking and followed by flushing the adherent cells with the culture medium or phosphate buffer saline or normal saline,
h) Adding fresh growth medium to the adherent Lympho-Myeloid Niches (LMN) with T cell or macrophage generation potential and continue the culture by incubating the culture vessel at 37° C. temperature with 5% CO2 concentration,
i) Maintaining the cells in Lympho-Myeloid Niches (LMN) for long term by removing the old growth or culture medium and then adding fresh growth or culture medium once in every 2-3 days or longer,
j) The resulting Lympho-Myeloid Niches (LMN) culture yields mixed population of T cells and macrophages in the absence of any cytokines or pure population of T cells upon culturing them in the presence of certain cytokines,
k) Collecting the non-adherent cells generated and detached from the niche by centrifugation and using for establishing secondary cultures and/or further expansion of the cells.

Yet another aspect of the present invention is to provide of method of generating macrophages and myeloid cells from Lympho-Myeloid Niches (LMN) comprising the steps of
a) Harvesting the cells from the Lympho-Myeloid Niches (LMN) using cell dissociating reagents,
b) Pelleting the detached cells and washing the cell pellet with isotonic buffer to remove the residual reagents,
c) subjecting the detached cells to fluorescent activated cell sorting or magnetic bead-based cell separation to obtain pure population of macrophages and myeloid cells which can be used for therapeutic, diagnostic, and other applications,
d) Adding Granulocyte Macrophage colony stimulating factor (GMCSF) either alone or along with Interleukin-4 in the cultures of Lympho-Myeloid Niches (LMN) or purified population of myeloid cells from the LMN for the generation of dendritic cells which can be used for therapeutic, diagnostic, and other applications.

As per another aspect of the present invention is to provide a method of generating T cell from Lympho-Myeloid Niches (LMN) comprising the steps of,
a) Obtaining pure population of T cells by adding T cell growth promoting cytokines such as IL-2 and/or IL-15 directly in the cultures of Lympho-Myeloid Niches (LMN),
b) collecting the T cells generated from the LMN by removing the culture medium and pelleting the suspended T cells and resuspending the T cells in fresh medium with IL-2 and/or IL-15 for further expansion and splitting the cells into new flasks as their number increases,
c) Continuing the T cell culture by replacing the old culture medium with fresh culture medium containing T cell growth promoting interleukins or cytokines as mentioned in step a) and b) once in every 2-3 days, and repeating the harvest as many times as possible until all the LMNs disappear from the culture,
d) Harvesting the pure T cells by centrifuging the T cells suspended in culture medium and suspending the cell pellet in normal saline or any other isotonic buffer or medium and use for therapeutic, diagnostic, drug discovery, drug testing, and animal model development purposes.

### DETAILED DESCRIPTIONS OF THE DRAWING

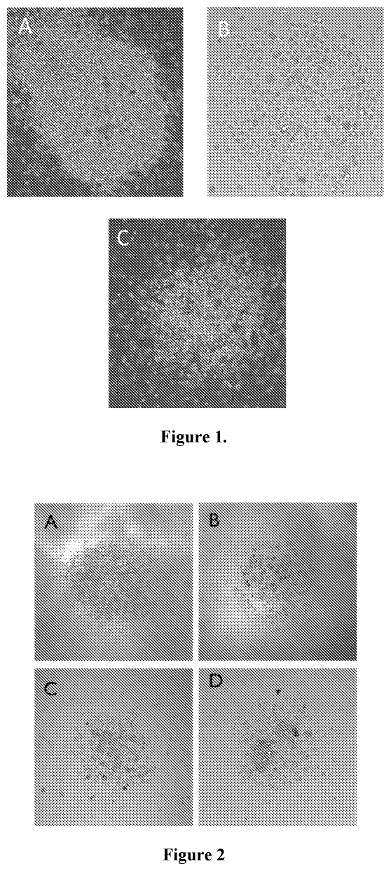

FIG. 1 illustrates the Niches of different morphology and different cellular composition observed in the cultures of primary human PBMCs in the laboratory.

FIG. 2 illustrates the individual niches obtained by limiting dilution approach. The 48 well plate was seeded with 50000 peripheral blood mononuclear cells in each well. On day 6, the media was replaced with fresh medium after the adherent cells were washed gently multiple times and then the media was replaced once in every 3 days afterwards. The images were captured on day 17 of culture.

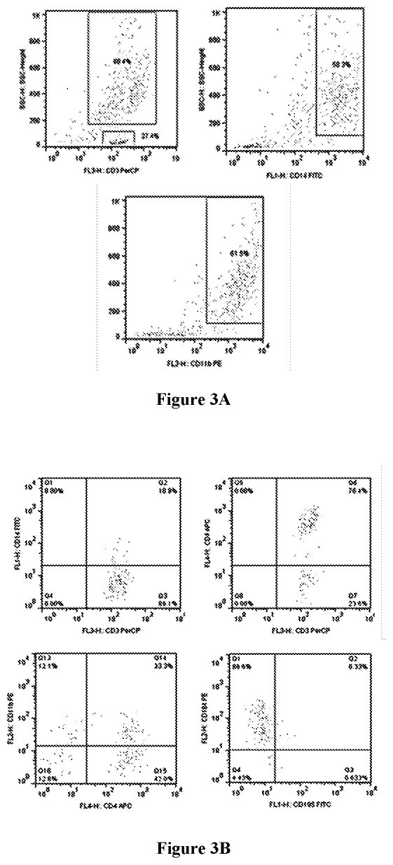

FIG. 3A illustrates the gating of T cells and macrophages, and analysis of macrophages with myeloid markers (CD14 and CD11b).

FIG. 3B illustrates the analysis of the gated T cells with T cell markers (CD3, CD4, CCR5 and CxCR4).

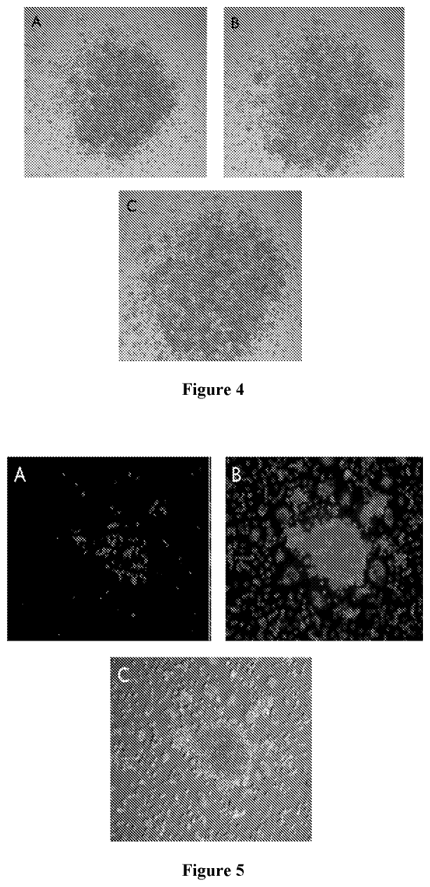

FIG. 4 illustrates the increase in the size of the niche over a period of time.

FIGS. 4A, 4B and 4C illustrates the selected images from the series of time lapse images captured over a period of 76 hours. The diameter of the niche increased from while comparing the starting frame (A) and the ending frame (C).

FIG. 5 illustrates the BrDU exposed cells stained with anti-BrDU antibody, nuclear stain DAPI and the wheat agglutinin. BrDU incorporated nuclei stained green with anti-BrDU antibody conjugated with FITC, all the nuclei were stained blue with the DAPI stain, the cell membrane was stained with red fluorescent Wheat germ agglutinin conjugate.

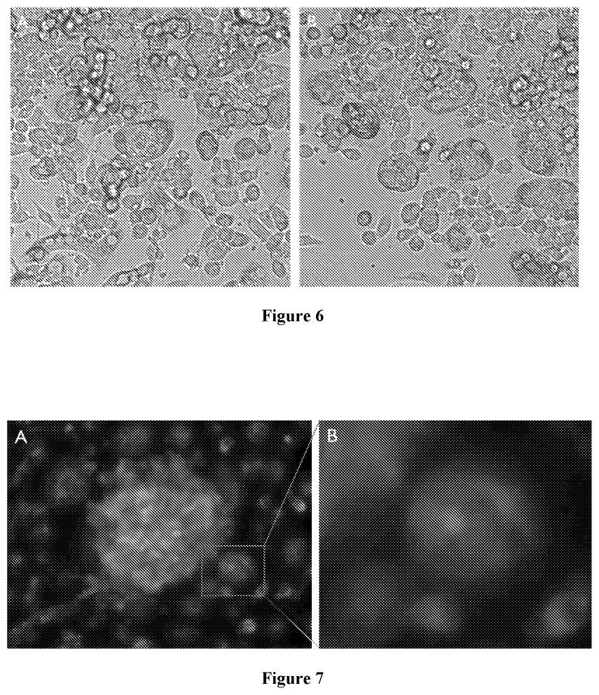

FIG. 6 illustrates the heterogeneous nature of cells in the niche. FIGS. 6A and 6B illustrates the images captured from different niches. Red arrow shows the large cells that are harboring small cells inside with a clear well-marked boundary.

FIG. 7 illustrates the BrDU labelling of the niche showing a Lymphocyte harboring macrophages (LHM) containing large number of T cells undergoing DNA synthesis, which appear to be the small cells observed in the light microcopy.

FIG. 7A illustrates the Niche showing BrDU incorporation and DNA synthesis.

FIG. 7B illustrates an individual Lymphocyte harboring macrophages (LHM) containing large number of small T cells undergoing DNA synthesis.

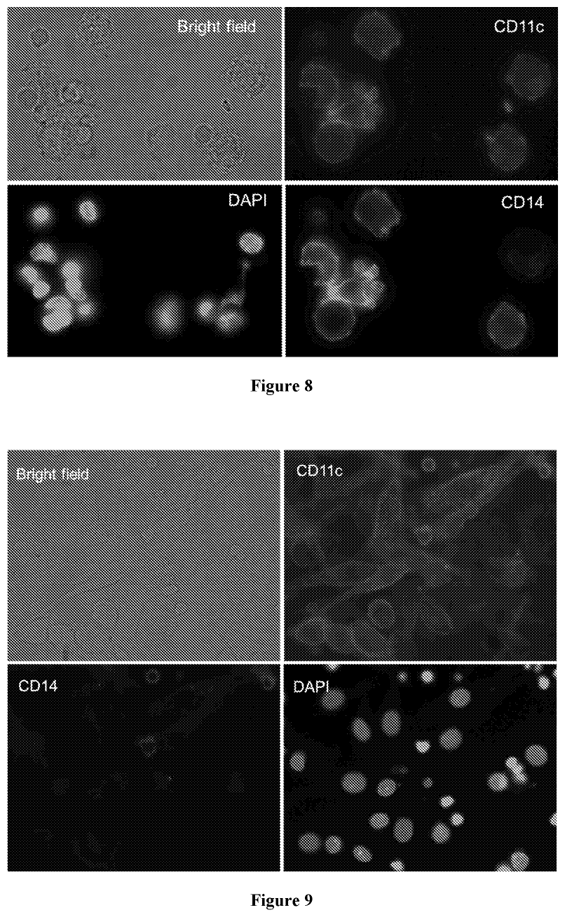

FIG. 8. Expression of myeloid markers CD11c and CD14 on the myeloid cells generated from the LMN in the absence of any GMCSF. Morphological features and expression of markers indicate these cells are monocytoid cells.

FIG. 9. Expression of myeloid markers CD11c and CD14 on the myeloid cells generated from the LMN in the presence of any GMCSF. Morphological features and expression of markers indicate these cells are dendritic or macrophage type of cells.

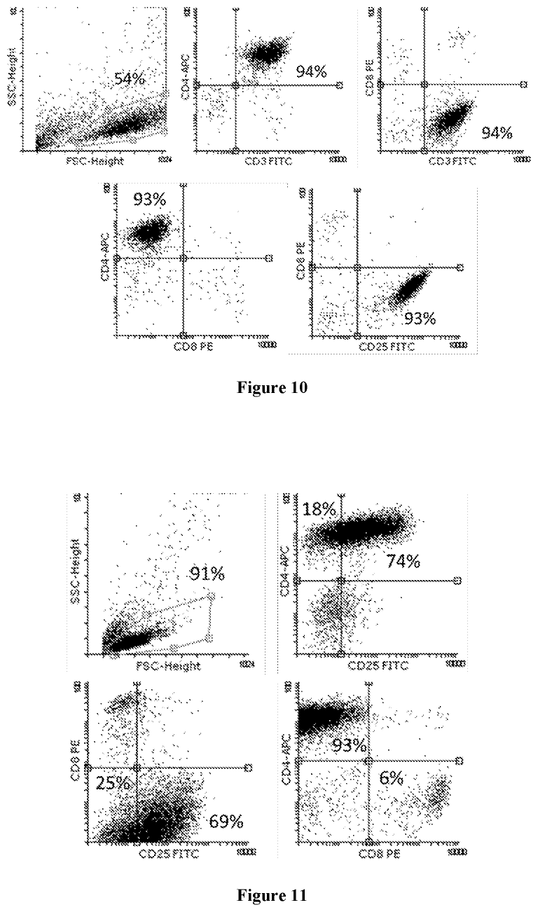

FIG. 10. Characterization of T cells generated from the LMN in the presence of Interleukin-2. The cell population is predominantly CD3+CD4+ T cells and only a small percentage of CD3+CD8+ T cells. All of these CD3+CD4+ T cells are also positive for CD25.

FIG. 11. Characterization of T cells generated from the LMN in the presence of Interleukin-15. The cell population is predominantly CD3+CD4+ T cells and only a small percentage of CD3+CD8+ T cells which is higher than the fraction generated using IL-2. This CD3+CD4+ T cells contained a larger fraction of CD4+CD25+ cells and a smaller fraction of CD4+CD25− cells.

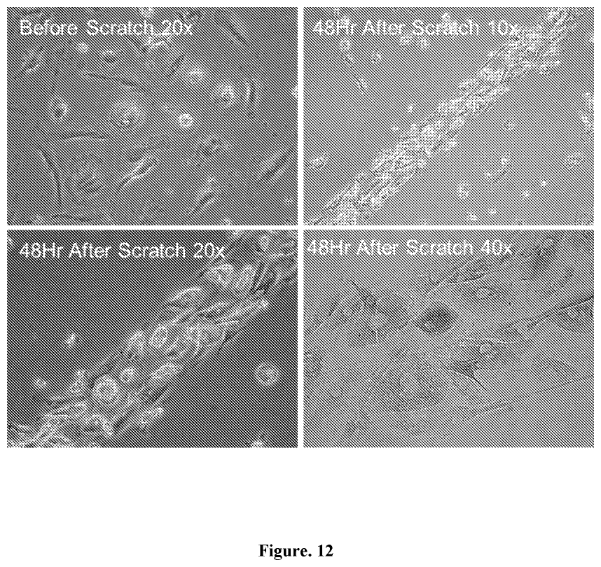

FIG. 12. showing the effect of Macrophages in scratch assay

### DETAILED DESCRIPTION OF THE INVENTION

The present invention describes a “Lympho-Myeloid Niches (LMN)” and novel method of generating Lympho-Myeloid Niches (LMN) from peripheral blood mononuclear cell (PBMC) obtained from human blood. The present invention also relates to a novel method of generating Lympho-Myeloid Niches (LMN). The present invention also relates to method of generating macrophages and myeloid cells as well as novel method of generating T cell from Lympho-Myeloid Niches (LMN).

In order that the present disclosure can be more readily understood, certain terms are first defined. As used in this application, except as otherwise expressly provided herein, each of the following terms shall have the meaning set forth below. Additional definitions are set forth throughout the application.

As used herein, the indefinite articles “a” or “an” should be understood to refer to “one or more” of any recited or enumerated component.

The terms “about” or “comprising essentially of refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of can mean within 1 or more than 1 standard deviation per the practice in the art. Alternatively, “about” or “comprising essentially of can mean a range of up to 10% (i.e., ±10%). For example, about 3 mg can include any number between 2.7 mg and 3.3 mg (for 10%).

It is understood that wherever aspects are described herein with the language “comprising,” otherwise analogous aspects described in terms of “consisting of and/or “consisting essentially of are also provided.

The term “Lympho-Myeloid Niches (LMN)” as used herein can be defined as an aggregation of cells including T cells and macrophages forming colony like structures, generated from peripheral blood mononuclear cells (PBMC), can perform de novo generation of large number of T Cells and myeloid cells, remain adherent to culture vessels. LMN are specialized cells mixture which are capable of generating large number of T cells and myeloid cells by simple and fast process due to its characteristics. The newly generated T cells and myeloid cells can remain adherent adjacent to LMN or released and remain non-adherent in the culture medium.

“Administering” refers to the physical introduction of an agent to a subject, using any of the various methods and delivery systems known to those skilled in the art.

Exemplary routes of administration for the T cells prepared by the methods disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the T cells prepared by the present methods is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

There are several types of T-cells, namely: Helper T-cells {e.g., CD4+ cells, effector T cells, T helper-1 or Th1, T helper-2 or Th2 cells, regulatory T cells), Cytotoxic T-cells (also known as TC, cytotoxic T lymphocyte, CTL, T-killer cell, cytolytic T cell, CD8+ T-cells or killer T cell), Memory T-cells ((i) stem memory TSCM cells, like naive cells, are CD45RO−, CCR7+, CD45RA+, CD62L+(L-selectin), CD27+, CD28+ and IL-7Ra+, but they also express large amounts of CD95, IL-2RP, CXCR3, and LFA-1, and show numerous functional attributes distinctive of memory cells); (ii) central memory TCM cells express L-selectin and are CCR7 and CD45RO+ and they secrete IL-2, but not IFNy or IL-4, and (iii) effector memory TEM cells, however, do not express L-selectin or CCR7 but do express CD45RO and produce effector cytokines like IFNy and IL-4), Regulatory T-cells (Tregs, suppressor T cells, or CD4+CD25+ regulatory T cells), Natural Killer T-cells (NKT), and Gamma Delta T− cells. T cells found within tumors are referred to as “tumor infiltrating lymphocytes” or “TIL.” B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

Cell “proliferation,” as used herein, refers to the ability of T cells to grow in numbers through cell division. Proliferation can be measured by staining cells with carboxyfluorescein succinimidyl ester (CF SE). Cell proliferation can occur in vitro, e.g., during T cell culture, or in vivo, e.g., following administration of a T cell therapy.

The term “genetically engineered,” “gene editing,” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which can either be obtained from a patient or a donor. The cell can be modified to express an exogenous construct, such as, e.g., a chimeric antigen receptor (CAR) or a T cell receptor (TCR), which is incorporated into the cell's genome.

A “therapeutically effective amount” or “therapeutically effective dosage,” as used herein, refers to an amount of the T cells or the DC cells that are produced by the present methods and that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of the T cells or

DC cells to promote disease regression can be evaluated using a variety of methods known to the skilled practitioner, such as in human subjects during clinical trials, in animal model systems predictive of efficacy in humans, or by assaying the activity of the agent in in vitro assays.

“Treatment” or “treating” of a subject refers to any type of intervention or process performed on, or the administration of one or more T cells prepared by the present invention to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission.

As described herein, any concentration range, percentage range, ratio range or integer range is to be understood to include the value of any integer within the recited range and, when appropriate, fractions thereof (such as one-tenth and one-hundredth of an integer), unless otherwise indicated.

The invention describes the novel Lympho Myeloid Niches (LMN) and method to generate them in the laboratory, and then to de novo generate T cells or myeloid cells from the peripheral blood mononuclear cells T cell precursors. The T cells generated using this approach can be used for therapeutic purposes.

The main embodiment of the present invention is to provide Lympho-Myeloid Niches (LMN) generated in vitro from PBMC which comprises of aggregation of cells and forming colony structures harbouring mainly T cells and macrophages, wherein LMN is characterized by the following features;
a) LMN provides a microenvironment, where the lymphoid and myeloid cells interact to each other and with the progenitor cells to de novo generate T cells and macrophages,
b) LMN are spontaneously generated in the cultures of peripheral blood mononuclear cells,
c) LMN are adherent to the culture vessels and remain intact upon removal of nonadherent cells,
d) LMN can de novo generate T cells and macrophages, where the newly generated T cells and macrophages migrate from the centre of the lympho myeloid niche towards the periphery,
e) LMN can generate T cells and macrophages by providing cytokines or growth factors that promote the T cells and macrophage proliferation, differentiation, and expansion.

As per one aspect, the present invention is to provide a method of generating Lympho-Myeloid Niches (LMN) comprising the steps of
a) Collecting blood from donor in a collection tube or bag containing anti-coagulant,
b) Recovering or isolating the PBMC from blood through Ficoll gradient centrifugation or any other method,
c) Resuspending the final cell pellet in culture medium containing 20% fetal bovine serum as growth medium,
d) Counting and seeding PBMC in cell culture flask or vessel,
e) Incubating the culture flask or vessel at 37° C. temperature in the presence of 5% CO2,
f) Observing the generation of adherent Lympho-Myeloid Niches (LMN) under the inverted microscope,
g) Removing nonadherent cells from culture flask or vessel by gently shaking and followed by flushing the adherent cells with the culture medium or phosphate buffer saline or normal saline,
h) Adding fresh growth medium to the adherent Lympho-Myeloid Niches (LMN) with T cell or macrophage generation potential and continue the culture by incubating the culture vessel at 37° C. temperature with 5% CO2 concentration,
i) Maintaining the cells in Lympho-Myeloid Niches (LMN) for long term by removing the old growth or culture medium and then adding fresh growth or culture medium once in every 2-3 days or longer,
j) The resulting Lympho-Myeloid Niches (LMN) culture yields mixed population of T cells and macrophages in the absence of any cytokines or pure population of T cells upon culturing them in the presence of certain cytokines.
k) The non-adherent cells generated and detached from the niche will remain in suspension can be collected by centrifugation and used for establishing secondary cultures and/or further expansion of the cells.

The blood can be collected from any individual (healthy donors, patients with certain diseases such as cancer or other diseases) and from any blood vessel. Blood can be collected in either in tubes with anticoagulants or blood collection bags with anticoagulant or through leukopheresis or apheresis procedure.

PBMC or monocytes is to be recovered from blood by ficoll-hypaque separation. To obtain large number of PBMC, leukopheresis procedure followed by ficoll-hypaque separation can be done. Any other manual or automated processes to obtain the blood or blood cells or blood mononuclear cells can also be used to obtain PBMC. Monocytes is to be obtained based on their adherence properties or magnetic bead based separation, or cell sorting or separation using any machine or instruments.

In one embodiment, culture media for present invention to generate Lympho-Myeloid Niches (LMN) can be selected from DMEM—Dulbecco's Modified Eagle Medium, Dulbecco's Modified Eagle Medium: Nutrient Mixture F-12, F10 Nutrient Mixture, Ham's F12 Nutrient Mixture, Media 199, Minimum Essential Media, RPMI Medium 1640, Opti-MEM I Reduced Serum Media, Iscove's Modified Dulbecco's Medium, serum free medium, or combinations thereof.

As per one embodiment, during process for preparing Lympho-Myeloid Niches (LMN), the serum is selected from Fetal Bovine Serum and human serum, serum supplements as alternative to serum, plasma preparations, platelet lysate preparations, albumin preparations, or any other combinations thereof.

As per one embodiment, the growth medium can include 5 to 30% human serum to improve the yield of LMN.

The LMN prepared as per above process contains the population of T cells and macrophages if cytokines are not used in the process. However, if the cytokines are used in the process, the LMN contains the population of pure T cells.

Yet another aspect of the present invention is to provide of method of generating macrophages and myeloid cells from Lympho-Myeloid Niches (LMN) comprising the steps of
a) Harvesting the cells from the Lympho-Myeloid Niches (LMN) using cell dissociating reagents,
b) Pelleting, the detached cells and washing the cell pellet with isotonic buffer to remove the residual reagents,
c) subjecting the detached cells to fluorescent activated cell sorting or magnetic bead-based cell separation to obtain pure population of macrophages and myeloid cells which can be used for therapeutic, diagnostic, and other applications,
d) Adding Granulocyte Macrophage colony stimulating factor (GMCSF) either alone or along with Interleukin-4 in the cultures of Lympho-Myeloid Niches (LMN) or purified population of myeloid cells from the LMN for the generation of dendritic cells which can be used for therapeutic, diagnostic, drug discovery, drug testing, and animal model development applications.

Cell Dissociating Reagent

Cell dissociating reagents are reagents used for removing the adherent cells attached to the surface of the culture vessel. It can be any enzymatic preparations such as Trypsin, accutase, collagenase, Dispase, etc or any chemical preparations such as EDTA, which help in detaching the adherent cells from the surface of the culture vessel so as to have the cells for further applications such as to use for any therapeutic or diagnostic purposes or for establishing secondary cultures.

Cell dissociating reagent for the present invention can be selected from the accutase, trypsin, collagenase, dispase, EDTA, or combinations thereof.

In one embodiment, culture media for present invention to generate macrophages and myeloid cells can be selected from DMEM—Dulbecco's Modified Eagle Medium, Dulbecco's Modified Eagle Medium: Nutrient Mixture F-12, F10 Nutrient Mixture, Ham's F12 Nutrient Mixture, Media 199, Minimum Essential Media, RPMI Medium 1640, Opti-MEM I Reduced Serum Media, Iscove's Modified Dulbecco's Medium, serum free medium, or combinations thereof.

As per one embodiment, during process for preparing macrophages and myeloid cells the serum is selected from Fetal Bovine Serum and human serum, serum supplements as alternative to serum, plasma preparations, platelet lysate preparations, albumin preparations, or any other combinations thereof.

As per one embodiment, seeding to be performed to the isolated or purified macrophages in cell culture vessels and culturing further to maintain them in the culture for long period of time.

As per one embodiment, the yield of macrophages and other myeloid cells in above process can be increased by adding growth factors such as GM-C SF (Granulocyte Macrophage Colony Stimulating Factor) and/or MCSF (Macrophage Colony Stimulating Factor), and/or interleukin-4 and or any other reagents to promote macrophage and myeloid cell generating and differentiation.

The macrophages or myeloid cells obtained from above process can further be used for any therapeutic, diagnostic, and other applications.

The macrophages generated from above method include conventional macrophages, lymphocyte harbouring macrophages (LHM), and other myeloid cells including dendritic cells.

As per one embodiment, growth factors such as GM-CSF (Granulocyte Macrophage Colony Stimulating Factor) and/or MCSF (Macrophage Colony Stimulating Factor) can be added in above process to promote dendritic cells, macrophage and other myeloid cell generation and differentiation from Lympho-Myeloid Niches (LMN) and to improve the yield.

As per one more embodiment macrophages, dendritic cells and other myeloid cells can be activated by adding cytokines such as interleukin-12, interferon gamma, lipopolysaccharide, interleukin-2, CD40L, OX40 and other activating reagents in the culture.

The macrophages, dendritic cells and/or myeloid cells obtained from above process can further be used for any therapeutic, diagnostic, drug discovery, drug testing, and animal model development purposes.

As per another aspect of the present invention is to provide a method of generating T cell from Lympho-Myeloid Niches (LMN) comprising the steps of,
a) Obtaining pure population of T cells by adding T cell growth promoting cytokines such as IL-2 and/or IL-15 directly in the cultures of Lympho-Myeloid Niches (LMN),
b) collecting the T cells generated from the LMN by removing the culture medium and pelleting the suspended T cells and resuspending the T cells in fresh medium with IL-2 and/or IL-15 for further expansion and splitting the cells into new flasks as their number increases,
c) Continuing the T cell culture by replacing the old culture medium with fresh culture medium containing T cell growth promoting interleukins or cytokines as mentioned in step a) and b) once in every 2-3 days, and repeating the harvest as many times as possible until all the LMNs disappear from the culture,
d) Harvesting the pure T cells by centrifuging the T cells suspended in culture medium and suspending the cell pellet in normal saline or any other isotonic buffer or medium and use for therapeutic, diagnostic, drug discovery, drug testing, and animal model development purposes.

Splitting the cells means collection of the T cells generated from the cultures as mentioned above along with the culture medium in a centrifuge tube and pelleting it by centrifugation.

As per one embodiment, the non-adherent cells generated and detached from the Lympho-Myeloid Niches (LMN) in the absence of any cytokine or growth factor that remain in suspension can be collected by centrifugation and used for establishing secondary cultures and/or further expansion of the cells.

As per one embodiment, harvesting adherent cells of Lympho-Myeloid Niches (LMN) using enzyme preparations such as Trypsin, Accutase, etc or cell dissociating reagents such as EDTA or combination of both and use for establishing secondary cultures or use for any therapeutic, diagnostic, and other purposes.

As per one embodiment, collecting the non-adherent cells and then adding T cell activating reagent such as anti-CD3 antibody, anti-CD28 antibody, 41BB, OX-40 or any other reagent with T cell activating properties to these cells for 1-3 days in the presence of interleukins such as IL-2, IL-15, IL-7, IL-9, IL-17, IL-21 or any other lymphoid lineage promoting cytokines. As per preferred embodiment the activating agent can be selected from anti-CD3 antibody, anti-CD28 antibody, 41BB, OX-40.

Alternative to generating large number of T cells from the primary cultures of LMN, T cells can be generated by harvesting the non-adherent cells generated from primary cultures of LMN and establishing secondary cultures by seeding them in new culture vessel and then adding one or more interleukins such as IL-2, IL-15, IL-7, IL-9, IL-17, IL-21 or any other lymphoid lineage promoting cytokines in the secondary cultures of cells harvested from primary Lympho-Myeloid Niches (LMN) and then use for any therapeutic, diagnostic, and other purposes by collecting the T cells.

The collected T cells obtained from above process can further be used for any therapeutic, diagnostic or other purposes directly or following further manipulation such as introduction of Chimeric antigen receptor (CAR) to generate CAR T cells or any other manipulation.

As per one more embodiment, the generated T cells can be activated by adding the reagents such as anti-CD3 antibody, anti-CD28 antibody, 41BB, OX-40, Phytoheamaglutinins or PHA, Concanavalin (ConA), Superantigens, Lipopolysaccharide or LPS, Immunomodulatory agents, polyclonal antibodies, monoclonal antibodies, recombinant proteins, ligands of receptors, inhibitors or activators of pathways, cancer antigens, complements, and their combination thereof or any other T cell activating reagents to the primary adherent cultures of Lympho-Myeloid Niches (LMN) in the presence of interleukins such as IL-2, IL-15, IL-7, IL-9, IL-17, IL-21 or any other lymphoid lineage promoting cytokines.

In one embodiment, culture media for present invention to generate Lympho-Myeloid Niches (LMN) can be selected from DMEM—Dulbecco's Modified Eagle Medium, Dulbecco's Modified Eagle Medium: Nutrient Mixture F-12, F10 Nutrient Mixture, Ham's F12 Nutrient Mixture, Media 199, Minimum Essential Media, RPMI Medium 1640, Opti-MEM I Reduced Serum Media, Iscove's Modified Dulbecco's Medium, serum free medium, or combinations thereof.

CO2 incubation: Cell culture medium require a suitable buffer to maintain the physiological pH. Many common mediums uses a sodium bicarbonate buffer system (2.0 g/L), and therefore requires a 5-10% CO2 environment to maintain physiological pH. It can be achieved by using the incubator equipped with CO2 regulation with or without 02 regulation. Some medium uses HEPES buffer to maintain the physiological pH. Maintaining the physiological pH is important for optimal cultures of Lympho-Myeloid Niches (LMN)

As per one embodiment, the T Cells which can be generated from Lympho-Myeloid Niches (LMN) are selected for CD4+ T cells, however, a small fraction of CD8+ T cells, gamma delta T cells, NK cells can also be obtained. As per one embodiment, T cells of any characteristics can be generated by this method by altering method accordingly. The activated and expanded effector CD4 and/or CD8 T cells are most commonly used for T cell based therapies through adoptive transfer method. The regulatory CD4 T cells are also used for treating autoimmune diseases or diseases with excessive unwanted immune response. The CD4 subset secreting IL-9 (Th-9) cells and Natural Killer T cells (NKT) are also used for some therapeutic purposes. The other T cells include Th17+ T cells which are targeted for treating autoimmune diseases. The memory T cells including central and peripheral memory T cells and the stem T cells are also being attempted for treating certain disease conditions.

As per one embodiment of present invention, the T cells obtained from the Lympho-Myeloid Niches (LMN) is used further by,

a. Introducing gene or DNA molecules in to T Cells,

b. Editing of the gene or DNA of Lympho-Myeloid Niches (LMN) and/or T cells

c. Adding antigen to Lympho-Myeloid Niches (LMN) and allowing them to present it to T cells,

d. Activating the Lympho-Myeloid Niches (LMN) using any one or more antigen and/or activation molecules or factors or their combination,

e. Collecting nonadherent cells in the Lympho-Myeloid Niches (LMN) cultures and establishing secondary cultures in new culture vessels or use it for the purposed mentioned in above steps,

f. Harvesting adherent macrophages or T cells or dendritic cells, stem cells using enzyme or cell dissociating reagents and then establishing secondary cultures in new culture vessels,

g. Use of the primary or secondary cultures for generating T cells and use of the T cells for the purposes mentioned in above steps,

h. Collecting the non-adherent and/or adherent cells of LMN including T cells and macrophages or dendritic cells and use it for the purpose of therapy,

i. Collecting the non-adherent and/or adherent LMN including T cells and macrophages or dendritic cells and use it for the purpose of developing in vitro or animal models or use it for any diagnostic purposes.

In one embodiment, the gene or DNA molecules which is to be introduced to Lympho-Myeloid Niches (LMN) are selected from, Phenylalanine hydroxylase (PAH), Cystic fibrosis conductance transmembrane regulator (CFTR), Beta hemoglobin (HBB), Oculocutaneous albinism II (OCA2), Huntingtin (HTT), Dystrophia myotonica-protein kinase (DMPK), Low-density lipoprotein receptor (LDLR); Connexin 26, apolipoprotein B (APOB), Neurofibromin 1 (NF1), Polycystic kidney disease 1 (PKD1) and polycystic kidney disease 2 (PKD2), Coagulation factor VIII (F8), Dystrophin (DMD), Methyl-CpG-binding protein 2 (MECP2), or any other gene associated with genetic disorders.

In one embodiment, the gene or DNA of Lympho-Myeloid Niches (LMN) is to be edited as per requirement or as per desired end use. Genes present in the genome can be edited for repairing a disease causing mutation or inducing resistance or reducing the risk of susceptibility to certain diseases using the gene editing tools such as CRISPR-Cas9, ZFN, and TALEN. For example, the natural T cell receptor and/or MHC genes can be mutated to use the allogenic cells for therapeutic purposes, mutated Beta globin or other disease causing genes can be repaired, CCR5 gene can be mutated to acquire resistance to HIV infection using gene editing approaches.

A variety of factors can activate Lympho-Myeloid Niches (LMN) which are including whole bacteria or bacterial-derived antigens (e.g. lipopolysaccharide, LPS), inflammatory cytokines (TNFa, IL1b, IL-12, etc), ligation of select cell surface receptors (e.g. CD40, OX40, PD1, PDL1) and viral products (e.g. double-stranded RNA, viral DNA, viral proteins, etc).

The macrophages can be harvested using the accutase or any other suitable enzyme or cell dissociation reagents. The adherent macrophages can be incubated with the enzyme or cell dissociation reagent. We found that the accutase can be used for optimal dissociation of the macrophages, however the other enzymes such as Trypsin, Dispase, Collagenase, Chymotrypsin or any extracellular matrix digesting enzyme can also be used. The cell dissociation reagents such as EDTA can also be used. In some cases the combination of both enzyme and dissociation reagent can be also used. The detached cells that come into the suspension can be pelleted by centrifugation.

As per one embodiment, magnetic bead based separation or cell sorting using manual or automated machine or instrument are other alternatives to obtain pure population of T cells or LMNs.

Another embodiment of the present invention is to provide a novel method of generating T cell. The T cells generated are primarily CD4 T cells, however, small percentage of CD8 T cells, gamma delta T cells, and NK cells are also generated.

As per one embodiment, the novel method of generating Lympho-Myeloid Niches (LMN) of present invention wherein the cytokines or interleukins is selected from interleukin-2 (IL-2), interleukin-15 (IL-15), interleukin-7 (IL-7), interleukin-4 (IL-4), interleukin-9 (IL-9), interleukin-10 (IL-10), interleukin-12 (IL-12), interleukin-17 (IL-17), interleukin-9 (IL-21), interleukin-1 (IL-1), Interferon, Tumor necrosis factor, ligands of activation receptor or co-receptor, Checkpoint inhibitors, Transforming growth factor, stem cell factor, Flt3 ligand, any other cytokines or interleukins or recombinant proteins or antibodies, or small molecules or plant extracts, and their combinations thereof.

In one embodiment the generated T cells can further be expanded and T cells can be increased by transferring split cells in to new flask and adding equal volume of fresh growth medium with serum, interleukin or cytokine, antibiotics and L-glutamine and then incubate at 5% CO2 in 37 C.

As per one embodiment, the method of generating Lympho-Myeloid Niches (LMN) and method of generating macrophages, myeloid cells and T cells from Lympho-Myeloid Niches (LMN) is analysed at different stages and recorded through different techniques like bright field or phase contrast microscopy, flow cytometer, fluorescent activated cell sorter, Giemsa-Wright staining, cell proliferation assays, and Time-lapse imaging. The method of all analysis and results are mentioned in Example 3.

As per one embodiment, in any of the process mentioned above, further antibiotic can be added and is selected from penicillin, ampicillin, bacitracin, streptomycin, gentamicin and ciprofloxacin, amphotericin, imidazone preparations, or combinations thereof. The antibiotic can be added to prevent infection to the LMN or macrophages or myeloid cells or T cells generated during the process of present invention.

The present invention also describes its application for developing novel cell based therapies or gene therapies or gene edited therapies for the treatment of various disease conditions using the Lympho-Myeloid Niches (LMN), and/or the T cells or myeloid cells generated from Lympho-Myeloid Niches (LMN) or their culture, use of these cells for gene therapies or gene edited therapies including Chimeric Antigen Receptor (CAR) T cell therapies or other adoptive T cell immunotherapies, for understanding any disease pathophysiology, creating novel animal models, development of novel drugs, developing vaccines or diagnostics or treatment including but not limited to Human immunodeficiency virus, cancer, Diabetic foot ulcers, non-healing wounds, autoimmune diseases, infectious diseases, etc.

Diabetic Foot ulcers are one of the most common complication of poorly controlled diabetes. It results due to breakage of skin tissue and exposing the cell layers underneath. These ulcers are most common under the big toes and the balls of the feet, and they are the most common cause of nontraumatic lower extremity amputations. and the risk of lower extremity amputation is 15 to 46 times higher in diabetic individuals than the non-diabetic individuals.

Incidence of non-healing wound is also increasing worldwide. It is estimated that 6.5 million patients with non-healing chronic wounds are living in United States alone and about 1-2 percent of the population will suffer from chronic wound in their life time. A non-healing wound is generally defined as a wound that will not heal within four weeks and its cause is usually found in underlying conditions that have either gone unnoticed or untreated. These wounds have failed to progress through a timely normal sequence of repair by restoring anatomy and function of the area.

As per one embodiment, the present invention is also proving an application for use of LMN and/or macrophages or dendritic cells for the treatment of diabetic foot ulcer and other non-healing wounds in effective manner.

Macrophages produced from the Lympho-Myeloid Niche (LMN) showed the property of wound healing in the scratch assay. The Macrophages were detached from the LMN and seeded on to a 35 mm culture dish and the dish was subjected to scratch on the next day. The cells were imaged before and after the scratch and showed the cells were able to migrated to the scratched area efficiently. The results are shown in FIG. 12.

The following examples are included to demonstrate certain embodiments of the invention. It should be appreciated by those of skill in the art that the techniques disclosed in the examples below represent techniques discovered by the inventors, and may be considered to constitute modes for practicing certain embodiments of the invention. However, those of skill in the art should, in light of the present disclosure, appreciate that many changes can be made to the examples below without departing from the spirit and scope of the invention.

### EXAMPLE

### Example 1: Method of Generating Lympho-Myeloid Niches (LMN) from PBMC

a) Blood was collected from donor in a collection tube or bag containing anti-coagulant,
b) PBMC were isolated from blood through Ficoll gradient centrifugation or any other method,
c) final cell pellets were resuspending in Culture medium containing 20% fetal bovine serum as growth medium,
d) seeding was done in PBMC in cell culture flask or vessel,
e) culture flask or vessel was incubated at 37° C. temperature in the presence of 5% CO2,
f) generation of adherent Lympho-Myeloid Niches (LMN) was observed under the inverted microscope,
g) nonadherent cells were removed from culture flask or vessel by gently shaking and followed by flushing the adherent cells with the culture medium,
h) fresh growth medium was added to the adherent Lympho-Myeloid Niches (LMN) with T cell or macrophage generation potential and continue the culture by incubating the culture vessel at 37° C. temperature with 5% CO2 concentration,
i) ells in Lympho-Myeloid Niches (LMN) was maintained for long term by removing the old growth or culture medium and then adding fresh growth or culture medium once in every 2-3 days or longer,

The resulting Lympho-Myeloid Niches (LMN) culture yields mixed population of T cells and macrophages in the absence of any cytokines or pure population of T cells upon culturing them in the presence of certain cytokines.

The non-adherent cells generated and detached from the niche will remain in suspension was collected by centrifugation and used for establishing secondary cultures and/or further expansion of the cells

### Example 2: Generation of Macrophages and Myeloid Cells from Lympho-Myeloid Niches (LMN)

a) Lympho-Myeloid Niches (LMN) prepared in Example 1, were used for further processing by removing macrophages from the Lympho-Myeloid Niches (LMN) using cell dissociating reagents,
b) The detached cells were Pelleted and washed with isotonic buffer solution to remove the residual reagents,
c) The detached cells were washed using cell culture medium or any isotonic buffer or solution,
d) detached cells were further subjected to fluorescent activated cell sorting or magnetic bead-based cell separation to obtain pure population of macrophages and myeloid cells which can be used for therapeutic, diagnostic, and other applications.

### Example 3: Generation of T Cells from Lympho-Myeloid Niches (LMN)

a) Lympho-Myeloid Niches (LMN) prepared in Example 1, were used for further processing
b) pure population of T cells was extracted by adding T cell growth promoting cytokines such as IL-2, IL-15 directly in the cultures of Lympho-Myeloid Niches (LMN),
c) T cells were also generated from the secondary cultures by adding one or more interleukins such as IL-2, IL-15,
d) adherent cells of Lympho-Myeloid Niches (LMN) were harvested using enzyme preparations such as Trypsin,
e) activated T cells were generated by adding the T cell activating reagent such as anti-CD3 antibody, and anti-CD28 antibody for 1-3 days in the presence or absence of interleukins such as IL-2, IL-15,
f) old culture medium was replaced with fresh culture medium containing interleukins or cytokines once in every 2-3 days and splitting the cells into new flasks as their number increases,
g) cell pellet were suspended in fresh growth or culture medium and seeding in a new culture vessel or in the same culture vessel for further expansion by proving one or more interleukins such as IL-2, IL-15.

### Example 4: Observations During Generation of Lymphoid and Myeloid Cell Generation Niche, and the T Cell from the Fraction of Peripheral Blood Mononuclear Cells

4.1. Culturing Cells:

PBMC were obtained using ficoll gradient centrifugation. Briefly, the blood was diluted and overlaid on the ficoll solution. PBMC were cultured using RPMI-1640 medium supplemented with 20% fetal bovine serum and 10% human serum or plasma. Human plasma or serum can be optional for short term cultures and the niches formation can be seen even in the absence of human plasma or serum. Generation of LMN were observed in the cultures at the laboratory. Purpose was to to know whether these niches are aggregations of preexisting cells or originate from the de novo generated cells. Limited dilution seeding of PBMC was carried out to obtain single niches in a well in 48 well plate. Optimization of the cell concentration revealed that 25000-50000 cells were needed to get 1-2 niches in single well of 48 well plate in 10-20% of the wells.

Results of cultured cells were shown in FIGS. 1, 2, 4, 5, 6, 7, 8 and 9.

4.2: Analysis of Cell by Flow Cytometer

Harvested cells were centrifuged, suspended in RPMI-20%, counted, and 50 μl aliquots were incubated with fluorochrome-labelled antibodies for 20 minutes at room temperature in the dark. One ml of DPBS containing 1% FBS and 0.09% sodium azide (wash buffer) was added, tubes were briefly vortexed, and then 250 μl of fixative (9.25% formaldehyde plus 3.75% methanol) was added, followed by 3 ml of wash buffer. Following centrifugation, cells were washed for an additional time, then suspended in 1% paraformaldehyde and acquired on a FACS Calibur cytometer (BD Biosciences) using CellQuest software. Ten-to-fourty thousand events were acquired per tube, depending on the frequency of populations of interest. Analyses were carried out using the CellQuest and FlowJo programs. The following antibodies were purchased from BD Biosciences: anti-CD3 (SK7), anti-CD4 (SK3), anti-CD8 (SK1), anti-CD14 (MΦP9), and the isotype control IgG1 (40). Results were represented in FIGS. 3, 10 and 11.

4.3. BrDU Labelling of Cells

The cells were grown in 24-well plates as described above. Bromodeoxyuridine (BrdU) was added at a final concentration of 50 mM at days 5, 7, 10 or 14 of culture. Cells were incubated with BrdU for 18-48 hours. The cells were fixed using 70% ethanol for 30 minutes, followed by a PBS wash, incubation with 0.5% Triton X-100 in DPBS for 10 minutes to permeabilize the cells, and then a 1 hour incubation with 2N HCl to denature the DNA. The cells were washed twice using PBS pH 8.0 and incubated for 2 hours with Alexa Fluor 488-conjugated mouse anti-BrdU diluted 1:8 in DPBS supplemented with 0.5% Tween 20. Later, the cells were washed thrice in DPBS, and counterstained by incubation with 2.8 μM DAPI for 10 minutes. Again, the cells were washed twice and observed using a fluorescent microscope.

Results of BRDU labelling experiments are shown in FIGS. 5 and 7.

4.4. Fluorescent Microscopy

After culturing the cells with BrDU, fixation of the cell was performed using 70% ethanol for 30 minutes. Then it was followed by a PBS wash, incubation with 0.5% Triton X-100 in PBS for 10 minutes to permeabilize the cells, and 1 hour incubation with 2N HCl to denature the DNA. The wells were then washed twice using PBS pH 8.0 and incubated for 2 hours with FITC-conjugated mouse anti-BrdU (BD Biosciences) diluted 1:4, in DPBS supplemented with 0.5% Tween 20. Following 3 washes in PBS, the cells were counterstained by incubation with 2.8 mM DAPI (Molecular Probes.) for 10 minutes, washed and examined using a Zeiss Axio Observer Z1 microscope.

Results are shown in FIGS. 5, 7, 8 and 9.

4.5: Bright Field and Time-Lapse Imaging:

Bright field and time lapse images of macrophage cultures were taken using an Amscope digital camera attached to Magnus Invi inverted microscope.

Results of bright field imaging are shown in Figure, 2, 5, 6, 8 and 9. Results of time lapse imaging is shown in FIG. 4.

1. (canceled)

2. A method of generating Lympho-Myeloid Niches (LMN) in vitro, the method comprising the steps of:
a) collecting blood from donor in a collection means containing anti-coagulant;
b) recovering or isolating peripheral blood mononuclear cells (PBMC) from blood;
c) resuspending the final cell pellet in a culture medium containing 20% serum as growth medium;
d) counting and seeding PBMC in a cell culture container;
e) incubating the culture container at 37° C. temperature in the presence of 5% CO₂;
f) observing the generation of adherent Lympho-Myeloid Niches (LMN) under an inverted microscope;
g) removing nonadherent cells from the culture container by shaking and followed by flushing the adherent cells with any one of the culture medium, phosphate buffer saline, and normal saline and obtaining adherent Lympho-Myeloid niches;
h) adding fresh growth medium to the adherent Lympho-Myeloid Niches (LMN) with T cell and macrophage generation potential and continue the culture by incubating the culture container at 37° C. temperature with 5% CO₂ concentration;
i) maintaining the cells in Lympho-Myeloid Niches (LMN) by removing the old culture medium and then adding fresh culture medium once in every 2-3 days; wherein the resulting Lympho-Myeloid Niches (LMN) culture yields mixed population of T cells and macrophages in the absence of cytokines and pure population of T cells upon culturing them in the presence of certain cytokines; and
j) collecting the non-adherent cells generated and detached from the niche by centrifugation and using for establishing secondary cultures and further expansion of the cells.

3. The method of per claim 2, wherein the culture medium is selected from DMEM—Dulbecco's Modified Eagle Medium, Dulbecco's Modified Eagle Medium: Nutrient Mixture F-12, F10 Nutrient Mixture, Ham's F12 Nutrient Mixture, Media 199, Minimum Essential Media, RPMI Medium 1640, Opti-MEM I Reduced Serum Media, serum free medium suitable for animal cell culture, Iscove's Modified Dulbecco's Medium, mammalian cell culture medium, and combinations thereof.

4. The method of claim 2, wherein the serum is selected from Fetal Bovine Serum and human serum, serum supplements as alternative to serum, plasma preparations, platelet lysate preparations, albumin preparations, and combinations thereof.

5. A method of generating macrophage cells from Lympho-Myeloid Niches (LMN) in vitro, the method comprising the steps of:
a) harvesting the macrophage cells from the Lympho-Myeloid Niches (LMN) using cell dissociating reagents resulting in detached cells;
b) pelleting the detached cells by centrifugation and washing the cell pellet with isotonic buffer to remove the residual reagents;
c) subjecting the detached cells to any one of fluorescent activated cell sorting and magnetic bead-based cell separation to obtain pure population of macrophage cells which can be used for therapeutic, and diagnostic applications; and
d) adding Granulocyte Macrophage colony stimulating factor (GMCSF) along with Interleukin-4 in the cultures of Lympho-Myeloid Niches (LMN) for the generation of dendritic cells used for therapeutic, diagnostic, drug discovery, drug testing, and animal model development purposes.

6. The method of claim 5, wherein the generated macrophage cells are configured to be used for the treatment of non-healing chronic wounds including diabetic foot ulcers.

7. The method of claim 5, wherein the Lympho-Myeloid Niches (LMN), the macrophages and dendritic cells are configured to be used for therapeutic, diagnostic, drug discovery, biomarker identification, gene therapy, gene-edited therapy, and animal model development purposes including for treating cancers and autoimmune diseases.

8. A method of generating T cells from Lympho-Myeloid Niches (LMN) in vitro, the method comprising the steps of:
a) obtaining pure population of T cells by adding any one of the T cell growth promoting cytokines such as IL-2, IL-15, IL-7, and combination thereof directly in cultures of Lympho-Myeloid Niches (LMN);
b) collecting the T cells generated from the LMN by removing the culture medium and pelleting the suspended T cells and resuspending the T cells in fresh culture medium with serum and one of the cytokines IL-2, IL-15, and IL-7 for further expansion and splitting the cells into new flasks as the number of cells increases;
c) continuing the T cell culture by replacing the old culture medium with fresh culture medium containing the serum and T cell growth promoting cytokine as mentioned in step a) and b) once in every 2-3 days, and repeating the harvest as many times as possible until all the LMNs disappear from the culture; and
d) harvesting pure T cells by centrifuging the T cells suspended in the culture medium and suspending the cell pellet in any one of normal saline, phosphate buffer saline, and culture medium.

9. The method of claim 8, wherein the generated T cells from the LMN are configured to be useful for therapeutic, diagnostic, drug discovery, biomarker identification, gene therapy, gene-edited therapy and animal model development purposes including Chimeric antigen receptor (CAR) T cell therapy, off-the-shelf allogenic CAR T cell therapy, adoptive T cell therapy, T cell immunotherapy, T-cell transplantation, antigen specific T cell receptor identification, CCR-gene edited T cells for treating HIV infected patients, and development of humanized animal models by transplanting T cells.

10. The method of claim 8, wherein the culture medium is selected from DMEM—Dulbecco's Modified Eagle Medium, Dulbecco's Modified Eagle Medium: Nutrient Mixture F-12, F10 Nutrient Mixture, Ham's F12 Nutrient Mixture, Media 199, Minimum Essential Media, RPMI Medium 1640, Opti-MEM I Reduced Serum Media, any serum free medium suitable for animal cell culture, Iscove's Modified Dulbecco's Medium, mammalian cell culture medium, and combinations thereof.

11. The method of claim 8, wherein the serum is selected from Fetal Bovine Serum and human serum, serum supplements as alternative to serum, plasma preparations, platelet lysate preparations, albumin preparations, and combinations thereof.

12. The method of claim 8, wherein cytokines or interleukins is selected from interleukin-2 (IL-2), interleukin-15 (IL-15), interleukin-7 (IL-7), interleukin-4 (IL-4), interleukin-9 (IL-9), interleukin-10 (IL-10), interleukin-12 (IL-12), interleukin-17 (IL-17), interleukin-21 (IL-21), interleukin-1 (IL-1), Interferon, Tumor necrosis factor, ligands of activation receptor or co-receptor, Checkpoint inhibitors, Transforming growth factor, stem cell factor, Flt3 ligand, and combinations thereof.

13. The method of claim 5, wherein the cell dissociating reagent is selected from Trypsin, accutase, collagenase, Dispase, EDTA and their combinations thereof.

14. The method of claim 5, wherein the purification of the macrophages is configured to be cultured in the presence of GMCSF with Interleukin-4 for the generation of dendritic cells.

15. The method of claim 2, wherein the generated LMN is configured to:
a) provide a microenvironment, where lymphoid and myeloid cells interact to each other and with the progenitor cells to generate T cells and macrophages (PBMC);
b) be spontaneously generated in the cultures of peripheral blood mononuclear cells;
c) be adherent to the culture vessels and remain intact upon removal of nonadherent cells;
d) generate T cells and macrophages, where the newly generated T cells and macrophages migrate from the centre of the lympho-myeloid niche towards the periphery; and
e) generate T cells and macrophages by providing cytokines or growth factors that promote the T cells and macrophage proliferation, differentiation, and expansion.
